(19) United States
(12) Patent Application Publication
(10) Pub. No.: US 2002/0172672 A1
(43) Pub. Date: Nov. 21, 2002

(54) Serine protease and topical retinoid compositions useful for treatment of acne vulgaris and production of anti-aging effects

(76) Inventors: Miri Seiberg, Princeton, NJ (US); Stephen J. Wisniewski, Doylestown, PA (US); Gerard F. Cauwenbergh, Plainsboro, NJ (US); Stanley S. Shapiro, Livingston, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/023,388

(22) Filed: Dec. 17, 2001

Related U.S. Application Data
(63) Continuation of application No. 09/015,565, filed on Jan. 29, 1998.
(60) Provisional application No. 60/037,605, filed on Feb. 12, 1997.

(51) Int. Cl. A61K 38/48

(52) U.S. Cl. 424/94.64; 424/94.63

(57) ABSTRACT

This invention is related to methods for treating Acne Vulgaris and/or for producing anti-aging effects on the skin of a mammal, and compositions effective for the same. More specifically, the present invention is directed to the use of serine proteases, as the sole active in a composition effective for the treatment of Acne Vulgaris and/or for producing anti-aging effects on the skin of a mammal, or in combination with a retinoid compound in a composition effective for the same.

### CROSS-REFERENCE TO RELATED APPLICATION

This Application claims the benefit of U.S. Provisional Application No. 60/037,605 filed on Feb. 12, 1997, which is incorporated herein by reference in its entirety.

### FIELD OF THE INVENTION

This invention is related to methods for treating Acne Vulgaris and/or for producing anti-aging effects on the skin of a mammal, and compositions effective for the same. More specifically, the present invention is directed to the use of serine proteases, either alone or in combination with a retinoid compound in a pharmaceutical or cosmetic composition.

### BACKGROUND OF THE INVENTION

Acne Vulgaris is a disorder of the pilosebaceous unit that affects nearly all adolescents to some degree, as well as many adults. The initial lesion of the disease is believed to be due to hypercornification and hyperkeratinization of the infundibulum, a process that helps to transform the sebaceous follicle into a comedone. This disorganization of the epithelium may give rise to inflammatory lesions, as the infundibulum ruptures and sebum is introduced into the dermis.

Accordingly, traditional therapies are directed against the three major pathological processes which contribute to the development of Acne Vulgaris. Treatments such as topical retinoids work against the obstruction of the sebaceous follicle resulting from abnormal desquamation of the follicular epithelium. Hormonal agents target the androgen-stimulated increase in the production of sebum. Finally, antibiotics function to reduce and/or halt the proliferation of propionibacteria within the follicle which contribute to inflammation. Benzoyl peroxide, salicylic acid, and various cleansing agents are also employed for similar purposes. Topical retinoids are considered to be one of the most effective classes of comedolytic agents for the treatment of Acne Vulgaris, however their clinical efficacy is limited by their irritant effects.

Topical retinoids have also been used to produce anti-aging effects on the surface of mammalian skin. While they are known in the art as one of the most effective topical treatments available, these compounds are limited by their irritant effects.

It would be desirable to provide a method for treating Acne Vulgaris which is as effective as traditional acne therapies, but which is not associated with high levels of irritancy.

It would also be desirable to provide a method for producing anti-aging effects on the surface of mammalian skin which is as effective as retinoid treatments, but does not have the same irritant effects.

### SUMMARY OF THE INVENTION

In accordance with the present invention, we have found a method for treating Acne Vulgaris and/or for producing anti-aging effects on the skin of a mammal comprising, consisting essentially of, or consisting of topically applying to the skin of a mammal an effective amount of a first topically active agent comprising a protease.

In another embodiment of the present invention, we have found a method for treating Acne Vulgaris and/or for producing anti-aging effects on the skin of a mammal comprising, consisting essentially of, or consisting of topically applying to the skin of a mammal an effective amount of a first topically active agent comprising a protease in combination with a second topically active agent comprising a retinoid.

In yet another embodiment of the present invention, we have found a pharmaceutical or cosmetic composition comprising, consisting essentially of, or consisting of:

a) a first topically active agent comprising a protease; and

b) a second topically active agent comprising a retinoid.

The compositions and methods of this invention provide a unique, convenient means for treating Acne Vulgaris and/or for producing anti-aging effects on the skin of a mammal.

### BRIEF DESCRIPTION OF THE DRAWINGS

The file of this patent contains several drawings executed in color. Copies of this patent with said color drawing will be provided by the Patent and Trademark Office upon request and payment of the necessary fee.

The invention will be more fully understood and further advantages will become apparent when reference is made to the following detailed description of the invention and the accompanying drawings in which:

FIG. 1(a) is a representation which illustrates a cross-sectional view of the skin of a Rhino mouse one hour after treatment with fluorescently labeled trypsin.

FIG. 1(b) is a representation which illustrates a cross-sectional view of the skin of a Rhino mouse four hours after treatment with fluorescently labeled trypsin. FIG (c) is a representation which illustrates a cross-sectional view of the skin of a Rhino mouse four hours after treatment with fluorescently labeled trypsin, following 5 days of daily treatment with 1% (w/v) trypsin.

FIGS. 2(a) and 2(b) are representations which illustrate the histology of Rhino mouse skins processed with H&E staining, (a) untreated, and (b) treated daily with 0.1% (w/v) trypsin in GDL liposomes for five days.

FIGS. 3(a) and 3(b) are color representations which illustrate the cross-sectional view of Rhino mouse skins which were processed for paraffin sections and stained for elastin.

FIG. 3(a) is vehicle treated, and

FIG. 3(b) is trypsin treated.

FIGS. 3(c) and 3(d) are representations which illustrate the cross-sectional view of C57Bl/6 mouse skins which were processed for paraffin sections and stained for elastin.

FIG. 3(c) is vehicle treated, and

FIG. 3(d) is trypsin treated.

FIGS. 4(a-c) are representations which illustrate the histology of Rhino mouse skins processed with H&E staining, (a) treated with 0.0005% (w/v) all-trans retinoic acid, (b) treated with 0.005% (w/v) trypsin, and (c) treated with both 0.0005% (w/v) all-trans retinoic acid and 0.005% (w/v) trypsin.

FIGS. 5(a) and 5(b) are representations which illustrate the cross-sectional view of the TUNEL-stained skin tissue of a vehicle treated Rhino mouse.

FIGS. 5(c) and 5(d) are representations which illustrate the cross-sectional view of the TUNEL-stained skin tissue of a Rhino mouse treated with trypsin.

FIG. 6 is a representation which illustrates the profile of gene expression of trypsin treated Rhino mouse skins at various concentrations of trypsin as detected by Reverse Transcription-Polymerase Chain Reaction (“RT-PCR”).

### DETAILED DESCRIPTION OF THE INVENTION

As used herein “(w/v)” shall mean grams of a given component per 100 ml of the total composition.

Topically active agents suitable for use in the compositions of the present invention as the first topically active agent include proteases, which include, but are not limited to, serine proteases. Preferably, the first topically active agent is selected from trypsin, carboxypeptidase-Y, protease IV, subtilysin, or mixtures thereof. The protease of choice is trypsin. Preferably, the protease is present in an amount, based upon the total volume of the composition of the present invention, of from about 0% (w/v) to about 5% (w/v), and more preferably from about 0.01% (w/v) to about 1% (w/v).

While not wishing to be bound by any theory, it is believed that the first topically active agent of the present invention treats the hyperkeratinization associated with Acne Vulgaris and/or produces anti-aging effects on the skin. Though the first topically active agent can be used as the sole active ingredient in a composition for the treatment of Acne Vulgaris and/or to produce anti-aging effects on the skin, to more thoroughly treat Acne Vulgaris, the first topically active agent of the present invention can be combined with a second topically active agent.

Again, while not wishing to be bound by any theory, it is believed that said second topically active agent treats both the hyperkeratinization and the obstruction of the sebaceous follicle associated with Acne Vulgaris, while also producing anti-aging effects on the skin which are comparable to those produced by the first topically active agent. Thus, as evidenced by Example 6 herein, the first feature of combining said first and second topically active agents is that the resulting treatment attacks at least two of the pathological processes associated with Acne Vulgaris, while not sacrificing the anti-aging benefits of the first topically active agent.

A second feature of combining said first and second topically active agents is evidenced by Example 4 herein, which shows that combining said first topically active agent with said second topically active agent mitigates the irritant effect associated with said second topically active agent. Thus, the efficacy of treatment of Acne Vulgaris and/or signs of anti-aging effects on the skin are approximately the same with the treatments of the present invention as compared with treatments involving the second topically active agent alone, but the irritant effect normally associated with said second topically active agent is substantially reduced.

A third feature of combining said first and second topically active agents is evidenced by Example 7 herein, which shows that combining said first topically active agent with said second topically active agent substantially reduces the time necessary for product efficacy as compared to the use of the second topically active agent alone. Thus, the efficacy of treatment remains approximately the same as compared with treatments utilizing the second topically active agent alone, but the length of time required to see results normally associated with said second topically active agent is substantially reduced by combining said second topically active agent with said first topically active agent.

Topically active agents suitable for use in the compositions of the present invention as the second topically active agent include those compounds in the class of retinoids, which include, but are not limited to, retinoic acids, vitamin A alcohol, vitamin A aldehyde, retinyl acetate, retinyl palmitate, or other derivatives, analogs or mixtures thereof. The retinoid of choice is all-trans retinoic acid. Preferably, the retinoid is present in an amount, based upon the total volume of the composition of the present invention, of from about 0.0001% (w/v) to about 0.5% (w/v), and more preferably from about 0.001% (w/v) to about 0.025% (w/v).

If the delivery parameters of the first topically active agent so require, the pharmaceutical or cosmetic compositions of the present invention may preferably be further comprised of a pharmaceutically or cosmetically acceptable vehicle capable of functioning as a delivery system to enable the penetration of the topically active agent into the utriculus. While any commercially available vehicle for delivering the first topically active agent to the appropriate skin appendage, which in this case is the utriculus, is suitable for use as the pharmaceutically or cosmetically acceptable vehicle, liposomes are preferred. The liposomes are more preferably non-ionic and comprised of: (a) glycerol dilaurate or glycerol distearate; (b) compounds having the steroid backbone found in cholesterol; and (c) fatty acid ethers having from about 12 to about 18 carbon atoms, wherein the constituent compounds of the liposomes are in a ratio of about 53:10:22 to about 63:20:32, and preferably from about 55:12:24 to about 61:18:30, respectively. Liposomes comprised of glycerol dilaurate/cholesterol/polyoxyethylene-10-stearyl ether (“GDL”) are most preferred. Preferably the liposomes are present in an amount, based upon the total volume of the composition, of from about 10 mg/mL to about 100 mg/mL, and more preferably from about 25 mg/mL to about 50 mg/mL. A ratio of about 58:15:27, respectively, is most preferred. Suitable liposomes may preferably be prepared in accordance with the protocol set forth in Example 2, though other methods commonly used in the art are also acceptable.

The above described liposomal composition may be prepared by combining the desired components in a suitable container and mixing them under ambient conditions in any conventional high shear mixing means well known in the art for non-ionic liposomes preparations, such as those disclosed in Niemiec et al., “Influence of Nonionic Liposomal Composition On Topical Delivery of Peptide Drugs Into Pilosebacious Units: An In Vivo Study Using the Hamster Ear Model,” 12 Pharm. Res. 1184-88 (1995) (“Niemiec”), which is incorporated herein by reference in its entirety.

In alternative embodiments, the pharmaceutical or cosmetic composition of the present invention may be optionally combined with other ingredients such as moisturizers, cosmetic adjuvants, anti-oxidants, surfactants, foaming agents, conditioners, humectants, fragrances, viscosifiers, buffering agents, sunscreens, colorants, preservatives, and the like in an amount which will not destroy the liposomal structure, if present, in order to produce cosmetic or pharmaceutical products.

When used in combination with one another, the first and second topically active agents of the present invention can be applied to the skin of a mammal either simultaneously or at different times. For example, in a first instance, if daily treatment with the combination of the first and second topically active agents is desired, the first topically active agent can be administered in the morning and the second topically active agent can be administered in the afternoon. In a second instance, to serve as an example only, the second topically active agent can be administered in the morning and the first topically active agent can be administered in the afternoon. In a third instance, again, to serve as an example only, the first and second topically active agents can be administered together. In a fourth instance, serving only as an example, the first and second topically active agents can be administered on alternate days. Furthermore, in a fifth instance, serving only as an example, the treatments with the first and second topically active agents do not have to be given in a one-to-one dosage, so the first topically active agent can be administered for two days, while the second topically active agent is administered on the third day and so on. There are, of course, multiple variations of this fifth instance. The previous five examples are provided only to illustrate some of the many different treatment regimens possible with the methods of the present invention. It should be understood that these examples are not limiting in any way to the treatment methods of the present invention, and that many other treatment regimens are possible.

The pharmaceutical or cosmetic composition should be applied in an amount effective to treat Acne Vulgaris and/or produce anti-aging effects on the skin. As used herein “amount effective” shall mean an amount sufficient to cover the region of skin surface where treatment of Acne Vulgaris and/or production of anti-aging effects is desired. Preferably, the composition is applied to the skin surface such that, based upon a square cm of skin surface, from about 2 μl/cm²to about 8 μl/cm² of topically active agent is present when treatment of Acne Vulgaris and/or production of anti-aging effects on the skin is desired.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

### The Rhino Mouse System

The Rhino mouse has been used as an experimental acne model to screen topically active comedolytic and antikeratinizing agents as described in Sundberg, J. P., “The Hairless and Rhino Mutations, Chromosome 14,” Handbook of Mouse Mutations With Skin and Hair Abnormalities 291-312 (1994), which is incorporated herein by reference in its entirety. A recessive mutation on chromosome 14 results in a mouse with wrinkled skin devoid of body hair by age 25 days. At that time, the end of the first hair cycle, the follicular papillae fail to follow the regressing hair follicles and become isolated in the dermis. The papillae do not reassociate with the follicular epithelium to initiate a new hair follicle cycle. The upper remnants of the hair follicle are filled with sloughed, cornified cells and form utriculi with a small sebaceous gland at their base, resembling an open comedone. The rhino skin becomes progressively loose, forming folds and ridges, due to the expansion of the surface, secondary to abortive hair follicles filling with cornified debris. The utriculi progressively enlarge, forming pilary cists (pseudocomedones), which are dilated follicular infundibula filled with cornified debris.

RHJ/LE Hairless (“Rhino”) male mice, 5-7 weeks of age, were obtained from Jackson Laboratories (Bar Harbor, Me.), and treated as described in Mezick et al., “Topical and Systemic Effects of Retinoids on Horn-Filled Utriculus Size in the Rhino Mouse: A Model to Quantify “Antikeratinizing” Effects of Retinoids,” 83 J. Invest. Dermatol. 110-113 (1984) (“Mezick”), which is incorporated herein by reference in its entirety.

### Example 2

### Preparation of Topically Active Compositions

A sufficient amount of lyophilyzed trypsin, available from Sigma-Aldrich Corporation (St. Louis, Mo.), was mixed into a buffered aqueous solution of 0.05 M N-2-hydroxyethylpiperazine-N′-2-ethane sulfonic acid available from Life Technologies, Inc. (Gaithersburg, Md.) under the tradename “Hepes” such that the pH of the resulting solution was about 7.4 and the concentration of trypsin in the solution was about 2% (w/v). One volume of the resulting trypsin solution was then mixed with one volume of (5%) glycerol dilaurate/cholesterol/polyoxyethylene-10-stearyl ether liposomes in water, which was prepared by the methods described in Niemiec, in order to yield a 1% (w/v) concentration of trypsin in the resulting topically active composition. The glycerol dilaurate was available from International Specialty Products Van Dyke (Belleville, N.J.) under the tradename “Emulsynt GDL.” The cholesterol was available from Croda, Inc. (Parsippany, N.J.) under the tradename “Cholesterol VSP/NF.” The polyoxyethylene-10-stearyl ether was available from ICI Surfactants Americas (Wilmington, Del.) under the tradename “Brij 76.” The volume to volume ratio of trypsin to GDL liposome, respectively, was altered to produce various concentrations of trypsin liposomal compositions.

Retinoic acid compositions contained an ethanol/propylene glycol vehicle which comprised 70% (w/v) ethanol (ethyl alcohol, 200 proof) which was obtained from Quantum Chemicals Corporation (Tuscola, Ill.) and 30% (w/v) propylene glycol which was obtained from Fisher Scientific (Pittsburgh, Pa.). The all-trans retinoic acid used in the retinoic acid compositions was obtained from BASF Aktiengesellschaft (Ludwigshafen, Germany). The volume to volume ratio of all-trans retinoic acid to ethanol/propylene glycol vehicle, respectively, was altered to produce various concentrations of retinoic acid compositions.

### Example 3

### Delivery of Trypsin into Hair Follicles

About 100 μL of the topically active trypsin composition of Example 2 was applied to the dorsal side of each Rhino mouse of Example 1. The trypsin used in this composition was fluorescently-labeled with a protein fluorescent labeling kit available from Molecular Probes, Inc. in accordance with its accompanying protocol (1996). At one and four hours after the application of the fluorescent trypsin treatment, a 1 cm by 2 cm sample of the skin surface of each mouse was isolated from each mouse with scissors, fixed with a 10% buffered formalin solution having a pH of about 6.9-7.1 at 25° C. available from Stephens Scientific, then formed into a paraffin block according to well-known procedures, and examined with fluorescent microscopy according to well-known methods.

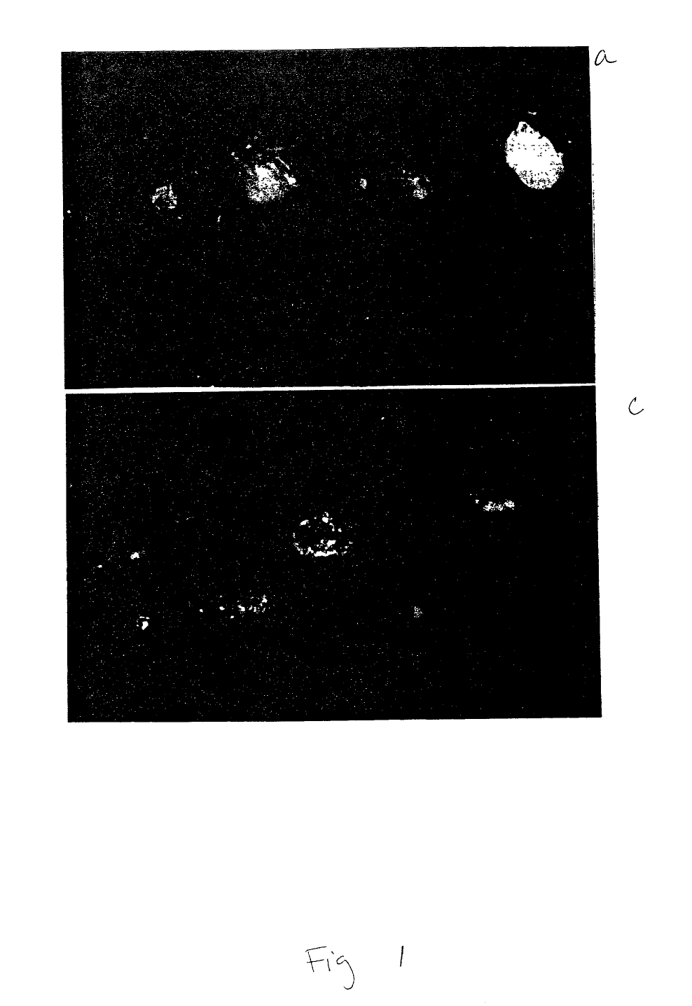

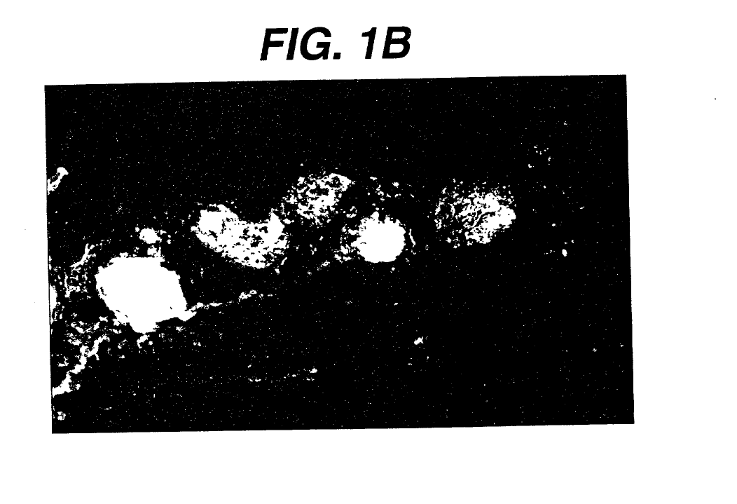

As shown in FIG. 1(a), almost all of the fluorescent labeling was found within the utriculi and sebaceous glands. The mice examined at the 1 hour interval (FIG. 1(a)) and the 4 hour interval (FIG. 1(b)) displayed identical histological staining patterns, with no additional skin penetration at the later time point. This observation suggests against a possible non-specific extracellular matrix digestion by the protease, which would have likely shown a deeper penetration of the fluorescent stain into the stratum corneum at the later time point.

This Example was repeated on similar Rhino mice of Example 1, with the exception that these mice were treated daily for 5 days with the 1% (w/v) trypsin composition of Example 2 prior to the fluorescent trypsin treatment. Four hours after the application of the fluorescent trypsin treatment on the fifth day, the skins of these mice were analyzed using similar fluorescent microscopic methods. As illustrated in FIG. 1(c), no major change was observed in the delivery route of the trypsin into the utriculi and sebaceous glands of the treated skins. However, the minimal staining at the outer portion of the stratum corneum of the trypsin-treated skins indicated some loss of barrier integrity. This loss of barrier integrity is reflected in the values for transepidermal water loss (“TEWL”) as described in Example 4 and Table 1 herein.

This Example shows that the application of a topically active composition containing trypsin to the skin surface of Rhino mice resulted in the delivery of the trypsin primarily to the utriculi and sebaceous glands, both after short term and long term use.

### Example 4

### Trypsin Treatment Reduces the Size of Utriculi but Does Not Induce Dermal Irritation

Rhino mice of Example 1 were topically treated with the trypsin compositions (0.001% (w/v)-1% (w/v)) of Example 2 once daily for five days. Animals were sacrificed at day 8 and image analysis was used to quantify the reduction in utriculi size. For image analysis, whole mount epidermis was processed and microscopic measurements were taken according to the methods described in Mezick as well as Bernerd et al., “The Rhino Mouse Model: The Effects of Topically Applied All-Trans Retinoic Acid and CD271 on the Fine Structure of the Epidermis and Urticuli Wall of Pseudocomedones,” 283(2) Arch. Dermatol. Res. 100-107 (1991) and Bouclier et al., “Quantification of Epidermal Histological Changes Induced by Topical Retinoids and CD271 in the Rhino Mouse Model Using a Standardizing Image Analysis Technique,” 4(2) Skin Pharmacol. 65-73 (1991) which are each incorporated herein by reference in their entirety. Empire Imagins Database version 1.1 was used on a Gateway 2000 P5-100 computer for capturing images. Image Pro Plus version 1.3 was used for measurements and Microsoft Excel version 5.0 for data processing. The mean utriculus diameter (μ) and the mean sebaceous gland size (μ²) were calculated for each treatment group (3 Rhino mice), using 5 random fields, two measurements per field, per animal. Percent reduction in utriculi diameter was calculated in accordance with the methods described in Finney, D. J., “Parallel Line Assays, Statistical Method in Biological Assay,” Charles Griffen & Company Ltd. 69-104 (1978) which is incorporated herein by reference in its entirety.

As shown in Table 1, trypsin induced a dose dependent reduction in utriculus size that reached a plateau at ˜0.1% (w/v) trypsin. A further increase in trypsin concentration did not result in more than 55% reduction of utriculus size relative to liposomal control. A small reduction in utriculus diameter was observed in the liposome vehicle alone. A single trypsin (1% (w/v)) treatment had no effect on utriculus size reduction when analyzed seven days later (not shown).

TABLE 1
Trypsin Induces a Dose Dependent Reduction in Utriculus Size

| | Utriculus Size | |
|---|---|---|
| | Reduction (%) vs. | |
| Treatment | Liposome Control | TEWL (g/m²h) |
| Trypsin 0.001% (w/v) | 26.85 ± 4.38 | 29.53 ± 3.68 |
| Trypsin 0.005% (w/v) | 19.58 ± 3.06 | 26.40 ± 1.77 |
| Trypsin 0.01% (w/v) | 33.08 ± 2.15 | 28.53 ± 2.18 |
| Trypsin 0.05% (w/v) | 43.84 ± 0.62 | 36.57 ± 2.07 |
| Trypsin 0.1% (w/v) | 50.67 ± 0.83 | 42.80 ± 4.33 |
| Trypsin 0.5% (w/v) | 54.31 ± 1.33 | 36.23 ± 1.24 |
| Trypsin 1.0% (w/v) | 54.85 ± 1.02 | 42.00 ± 1.14 |
| Liposome Vehicle | 13.2 ± 1.82* | 19.8 ± 1.14 |

*Percent of utriculus size reduction of the liposome vehicle treatment was calculated relative to the untreated control

To further characterize the effect of trypsin on the Rhino mouse skin, we measured the transepidermal water loss (“TEWL”) using an “Evaporimeter EPI” evaporimeter available from Servomed AB by first normalizing the evaporimeter with the ambient humidity and then placing the probe on the dorsal skin of the test subject at which point a reading of TEWL was taken.

As shown in Table 1, TEWL increased in a dose dependent manner, with a plateau reached at ˜0.05% (w/v) trypsin. This is approximately the same concentration for the maximal reduction in utriculi diameter. There was no correlation between TEWL increase and visual irritation. The minor scaling and erythema observed throughout these experiments were not dose dependent and remained low even at 1% (w/v) trypsin. Furthermore, the TEWL for trypsin treated mice was lower than that for retinoid treatment given alone.

Histological analysis of untreated, liposome control, and trypsin treated Rhino mice skins revealed major changes in the trypsin treated skins. H&E staining and histological analysis were performed using standard techniques as described in Sheehand and Hrapchak, 1980.

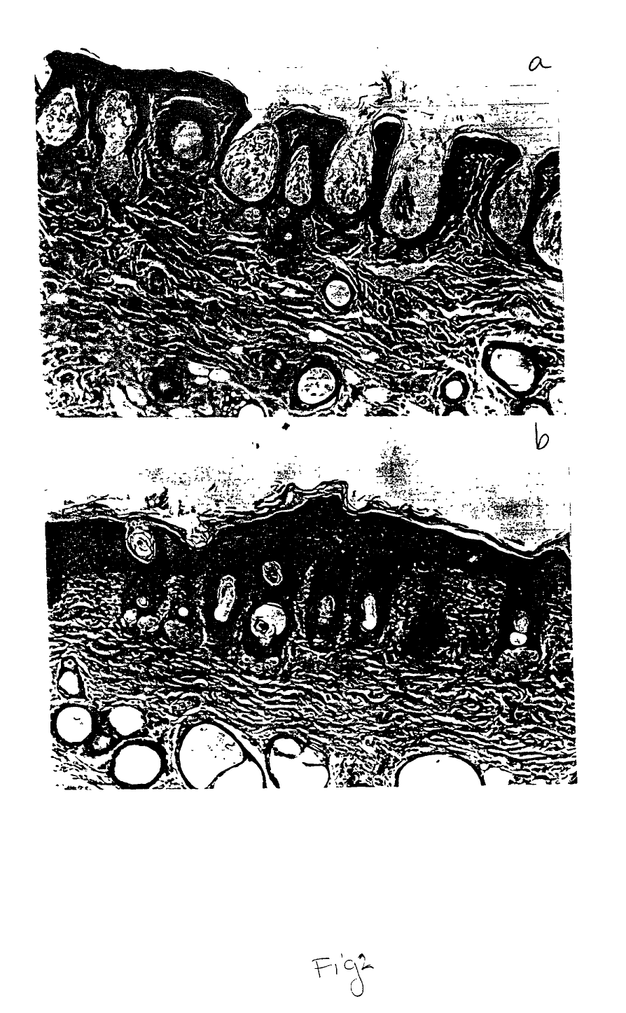

As shown in FIG. 2(b), the trypsin treated epidermis was hyperplastic with an increase in the number of cell layers of both the follicular epithelium and the epidermis when compared with the untreated epidermis shown in FIG. 2(a). Changes were observed mainly at the granular layer and the stratum corneum resulting in restored desquamation and improved skin structure. These epidermal changes are well-characterized markers for retinoid activity in vivo, and are associated with potential clinical efficacy. To further support the assertion that trypsin is unrelated to dermal irritation, FIG. 2(b) shows no inflammatory cells, which would normally be present in an irritation situation.

This example shows that trypsin causes a dose dependent reduction in the size of utriculi. A reduction in the size of the utriculi is associated with potential clinical efficacy of compositions for treating Acne Vulgaris. Therefore, this example further shows that trypsin is effective in the treatment of Acne Vulgaris. This example further shows that topical trypsin treatments do not induce skin irritation.

### Example 5

### Trypsin Treatment Results in Increased Skin Elasticity

Rhino mice of Example 1 which were treated with the trypsin composition of Example 2 showed a noticeable effect in skin elasticity. To quantitate this effect, a cutometer analysis was performed. We used a cutometer available from Acaderm (Menlo Park, Calif.), and employed the methods described in Couturaud et al., “Skin Biomechanical Properties: In Vivo Evaluation of Influence of Age and Body Site by a Non-Invasive Method,” 1 Skin Res. and Technol. 68-73 (1995) and Elsner et al., “Mechanical Properties of Human Forearm and Vulvar Skin,” 122 Br. J. Dermatol. 607-614 (1990) which are both incorporated herein by reference in their entirety. Suction was applied through a 2 mm aperture and the corresponding skin displacement and recovery after release of the negative pressure were measured. In human studies, an improvement in the ratios of deformation parameters Ua/Uf (skin fatigue, or total recovery from the load), Ur/Uf (biological elasticity, or elastic recovery after loading), and Ur/Ue (firmness, or improvement in the deformation resistance of the skin) indicates better tonicity and elasticity of the skin. The deformation parameters Ue, Uf, Ua, and Ur are dependent, in part, on skin thickness. Consequently, ratios were used for evaluation as described in Barel et al., “Suction Method for Measurement of Skin Mechanical Properties: The Cutometer,” Handbook of Non-Invasive Methods and the Skin 335-340 (1995) which is incorporated herein by reference in its entirety.

As shown in Table 2, trypsin treatment resulted in an increase in all of these parameters, which reflects improved skin elasticity. While variations between animals were significant, the increase in cutometric properties was consistent, and increased with time and length of treatment.

TABLE 2
Mechanical Properties of Trypsin Treated Rhino
Skin

| Biophysi- | Day 7 | | Day 12 | | Day 16 | |
|---|---|---|---|---|---|---|
| cal | Untreated | Trypsin | Untreated | Trypsin | Untreated | Trypsin |
| Parameter | Control | Treated | Control | Treated | Control | Treated |
| Ua/Uf | 0.541 ± 0.40 | 0.593 ± 0.09 | 0.656 ± 0.08 | 0.663 ± 0.10 | 0.429 ± 0.09 | 0.675 ± 0.03 |
| Ur/Ue | 0.408 ± 0.80 | 0.557 ± 0.21 | 0.242 ± 0.06 | 0.666 ± 0.24 | 0.243 ± 0.06 | 0.733 ± 0.18 |
| Ur/Uf | 0.300 ± 0.19 | 0.359 ± 0.22 | 0.370 ± 0.03 | 0.548 ± 0.11 | 0.204 ± 0.31 | 0.404 ± 0.08 |

To further study this elasticity effect, skin sections of Rhino mice from Example 1 treated with the trypsin composition of Example 2 were stained for elastin on paraffin sections in accordance with the methods set forth in Kligman, L. H., “Luna's Technique, A Beautiful Stain for Elastin,” 3(2) The Amer. J. of Dermatopathol. 199-200 (1981) which is incorporated herein by reference in its entirety.

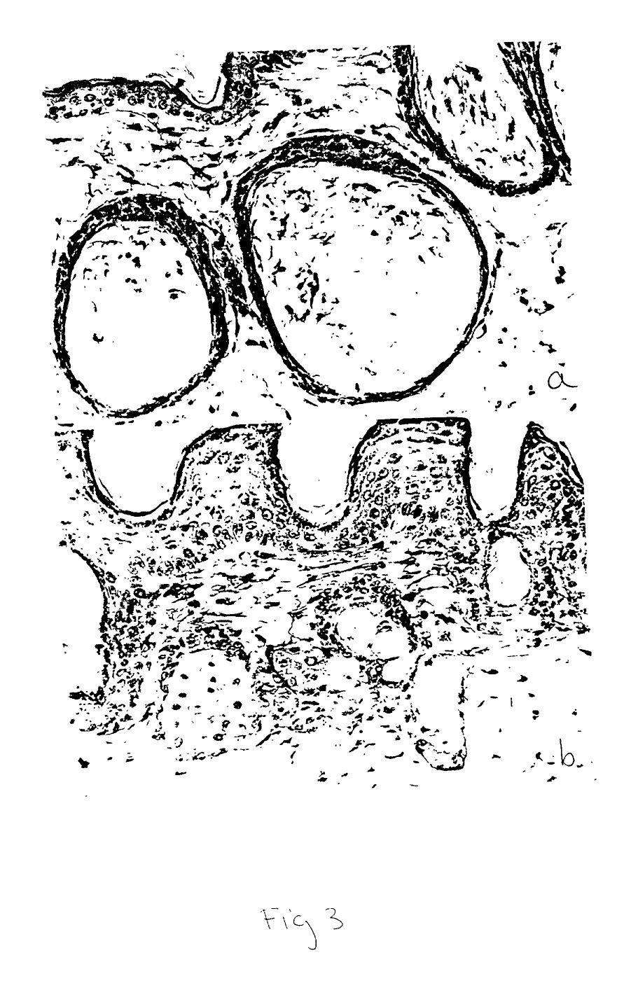

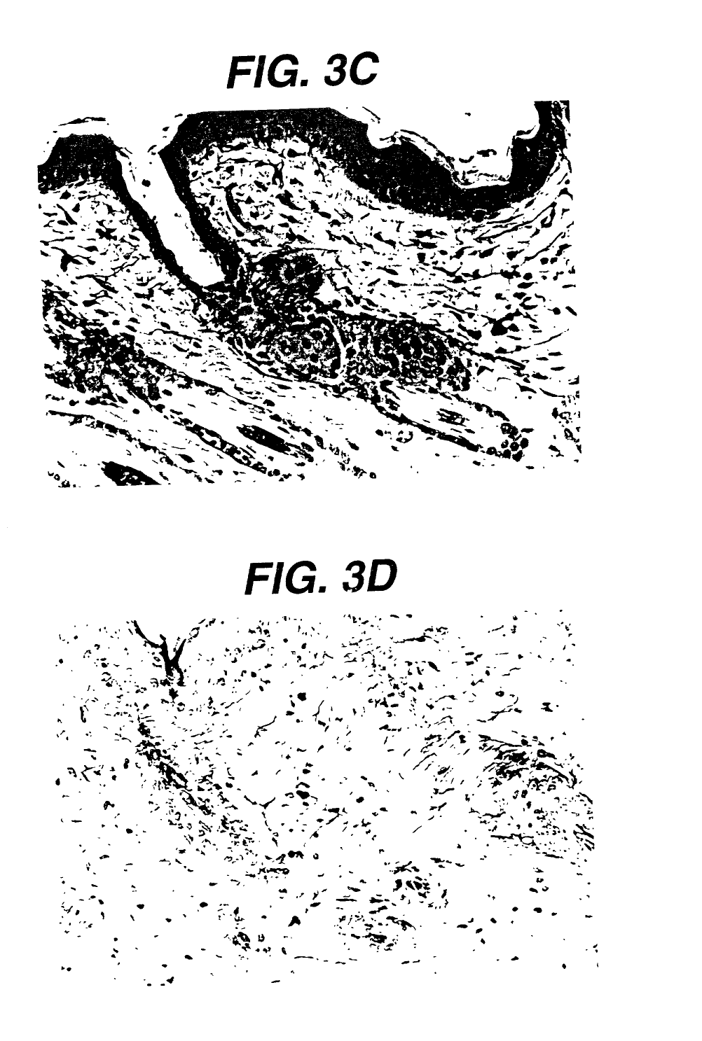

As shown in FIG. 3(b), elastin fibers (stained purple) were increased in thickness and density around the utriculi and the sebaceous glands of the trypsin treated Rhino mice when compared to the untreated mice of FIG. 3(a).

This same experiment was performed with C57Bl/6 mice which were obtained from Charles River Laboratories (Kingston, N.Y.) with similar results. FIGS. 3(c) and (d), the untreated and trypsin treated skins, respectively, show the results of elastin staining. Table 3 below shows the increase in skin mechanical parameters following the trypsin treatment.

TABLE 3
Mechanical Properties of Trypsin Treated C57B1/6 Skin

Day 16

| Biophysical | Untreated | Trypsin |
|---|---|---|
| Parameter | Control | Treated |
| Ua/Uf | 0.429 ± 0.14 | 0.675 ± 0.18 |
| Ur/Ue | 0.243 ± 0.21 | 0.7335 ± 0.02 |
| Ur/Uf | 0.204 ± 0.26 | 0.404 ± 0.1 |

This example shows that topical treatment with trypsin increases the elasticity of C57Bl/6 and Rhino mouse skins. Skin elasticity is a property associated with anti-aging. Therefore, this example further shows that trypsin imparts anti-aging effects to the surface of the skin.

### Example 6

### Trypsin Acts with a Mechanism Different from that of Retinoic Acid

The possible effect of trypsin on the sebaceous component of acne was evaluated using the hamster ear model system. Young golden Syrian hamsters, 45-55 grams upon arrival, were purchased from Charles River Laboratories (Wilmington, Mass.). The ventral side of the hamsters' right ears were treated daily with 10 μl of the trypsin composition of Example 2, five days a week for three weeks, while the left ears were used as untreated controls. As shown in Table 3, trypsin had no effect on the size of the sebaceous gland in this system.

TABLE 3
Effect of Trypsin on Size of Hamster Ear Sebaceous Gland

| | Sebaceous Gland | Percent Size |
|---|---|---|
| Treatment | Size (μ²) | Decrease (%) |
| Untreated | 99112.4 ± 2904.0 | N/A |
| Liposome Vehicle | 94698.9 ± 4997.1 | 4.45 (vs. untreated) |
| Trypsin 0.5% (w/v) | 95043.0 ± 4269.1 | −0.36 (vs. liposome vehicle) |

This example shows that trypsin had no effect on the size of the sebaceous glands in the hamster ear model system. It is well known that in this type of model, retinoids induce a dose dependent reduction in the size of the hamster ear sebaceous glands. Therefore, this example further suggests that trypsin functions with a mechanism different from that of retinoid compounds.

### Example 7

### Trypsin and Retinoic Acid Exhibit an Additive Therapeutic Effect

A first set of Rhino mice of Example 1 were treated with suboptimal doses of the trypsin composition of Example 2. As used herein, “suboptimal” is defined as levels of trypsin concentration below the optimum for utriculi size reduction as demonstrated in Example 4. A second set of Rhino mice of Example 1 were treated with suboptimal concentrations of the all-trans retinoic acid composition of Example 2. A third set of Rhino mice of Example 1 were treated with both suboptimal doses of the trypsin composition of Example 2 and the all-trans retinoic acid composition of Example 2. In this third set of Rhino mice, the trypsin and all-trans retinoic acid treatments were each administered daily, but at different times (i.e. trypsin in the morning and all-trans retinoic acid in the afternoon). Mice were sacrificed and their skins were examined histologically with the procedure set forth in Example 3.

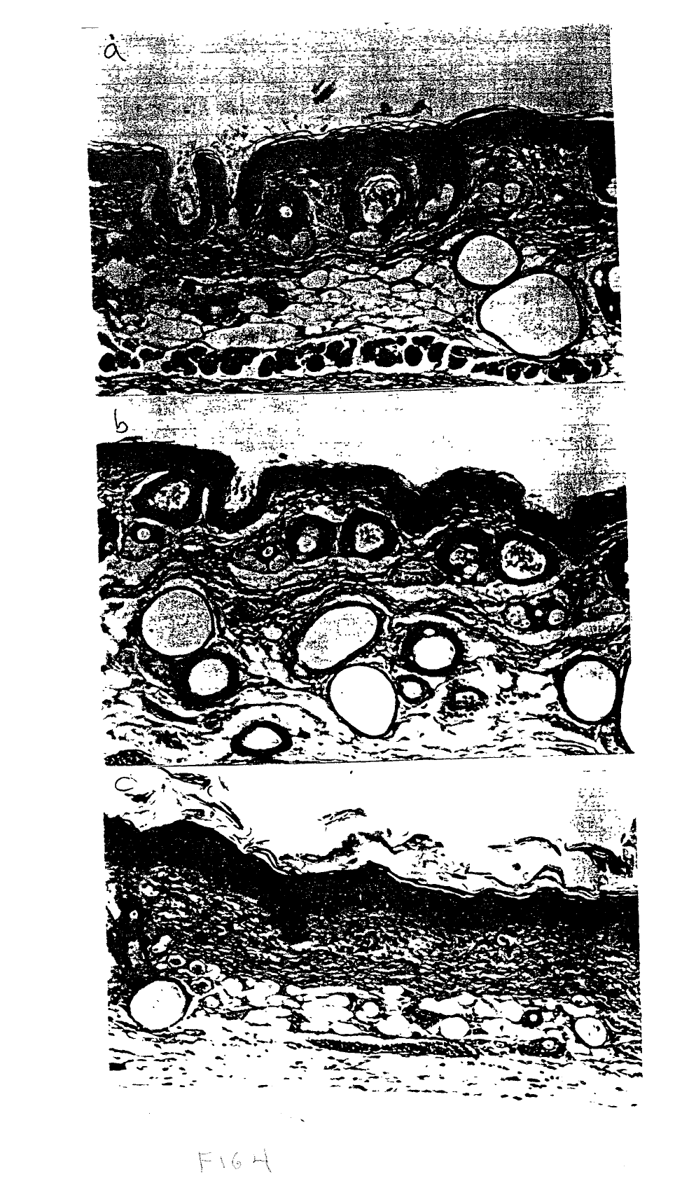

As shown in FIG. 4(c), Rhino mice treated with both the trypsin and all-trans retinoic acid compositions showed much improved desquamation when compared to the trypsin and all-trans retinoic acid treatments given alone (FIGS. 4(a&b)), though the treatments given alone showed marked inprovement over the untreated skin (FIG. 2(a)). Furthermore, the histological analysis revealed far fewer open utriculi in the surface of treated skins than either treatment given alone.

This example shows that a combined treatment of trypsin and all-trans retinoic acid produces an additive effect on skin surface characteristics such as the number of open utriculi, which means that these compositions are effective in the treatment of Acne Vulgaris.

### Example 8

### Trypsin Eliminates PCD in the Follicular Epthelium

Rhino mice of Example 1 were treated daily with a 0.1% (w/v) trypsin composition of Example 2 for five days and sacrificed at day eight.

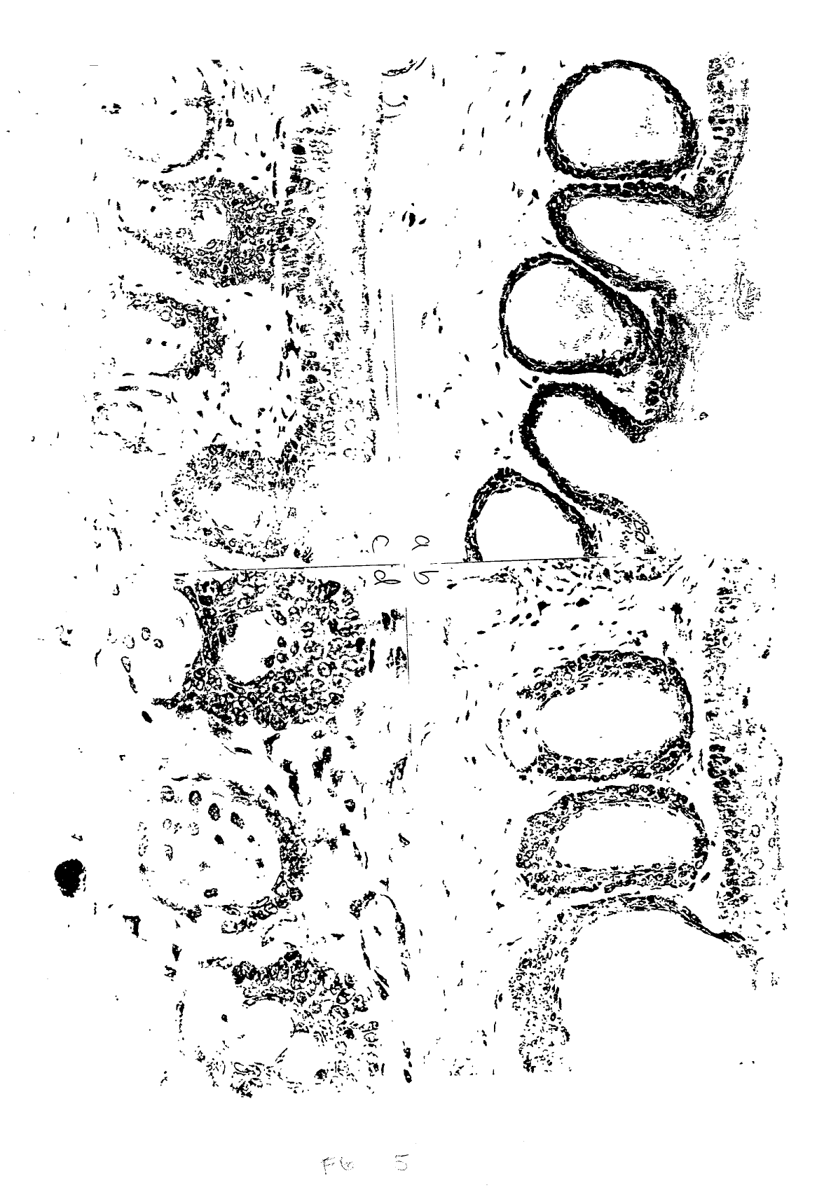

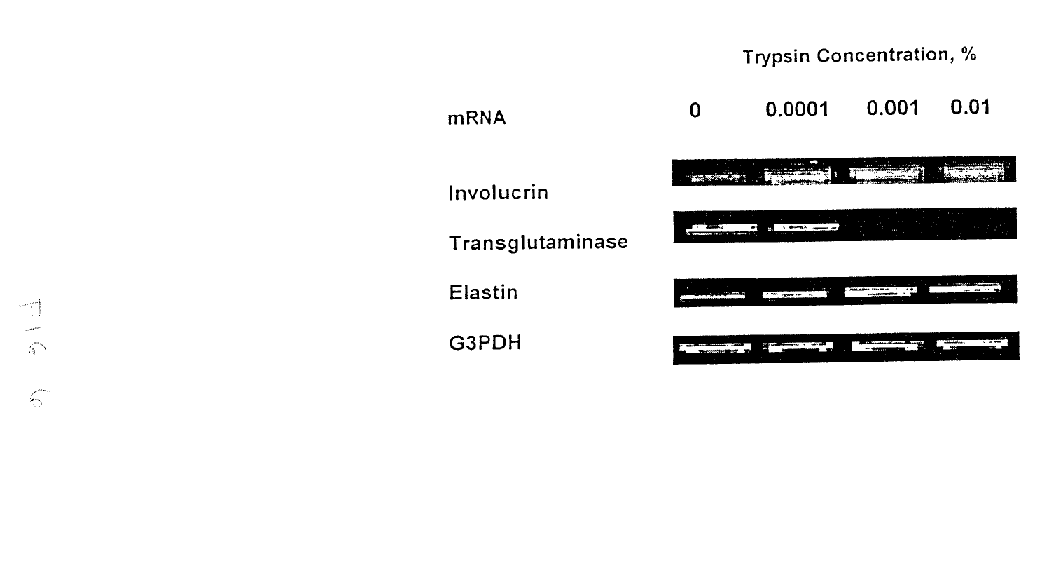

1 cm by 2 cm samples of the skins of untreated, vehicle treated, and trypsin treated mice were obtained via the procedure set forth in Example 3 then analyzed using a TdT-mediated dUTP-biotin nick end labeling (“TUNEL”) stain procedure as disclosed in Gavrieli et al., “Identification of Programmed Cell Death in situ Via Specific Labeling of Nuclear DNA Fragmentation”, 119 Jl. Cell Biology 493-501 (1992) (“Gavrieli”). During this procedure, the prepared skin sections were stained using an “ApopTag™ Plus In Situ Apoptosis Detection Kit” available from Oncor, Inc. as specified in the “ApopTag™ Plus In Situ Apoptosis Detection Kit” protocol by Oncor, Inc. (February 1995), which is based upon the labeling of fragmented DNA ends as described in Gavrieli. FIGS. 5(a-d) show a histological analysis wherein the stain has a peroxidase end point (brown) and a methyl green counter-stain. The resulting representations of this are provided in FIGS. 5(a&b) which are vehicle treated and FIGS. 5(c&d) which are trypsin treated.

As illustrated in FIGS. 5(a-d), the TUNEL-stained samples defined apoptotic cells by both morphology (condensed or fragmented nuclei and cytoplasm or apoptotic bodies) and by the color of its stain (fragmented DNA within the condensed nuclei were stained brown). As shown in FIGS. 5(a&b), TUNEL staining revealed an unusually high level of apoptotic bodies in the follicular epithelium. Trypsin treatment resulted in the elimination of all the apoptotic bodies within the follicular epithelium and the restoration of programmed cell death (“PCD”) at the granular layer (FIGS. 5(c&d)) as epidermal differentiation was restored.

This example suggests that trypsin could restore the balance between cell death and proliferation within the follicular epithelium and within the epidermis. One of the contributing pathological processes of Acne Vulgaris is hyperkeratinization, which may result from a shift in this balance. Therefore, this example further shows that the ability of trypsin to restore the proper balance in epithelial cell death and proliferation may be a factor in its ability to treat Acne Vulgaris.

### Example 9

### Tzypsin Induces Changes in Gene Expression

Rhino mice of Example 1 were treated daily with trypsin compositions (0% (w/v), 0.0001% (w/v), 0.001% (w/v), and 0.01% (w/v)), as prepared in Example 2, for five days and sacrificed at day eight. The skins of vehicle treated mice and trypsin-treated mice were obtained as described in Example 3, then their total RNAs were extracted using “RNA Stat-60” reagent available from Tel-Test “B,” Inc. as described in Chomczymski, “Single Step Method of RNA Isolation By Acid Guanidinium Thiocyanate-phenol-chloroform extraction,” 162 Anal. Biochem. 156-59 (1987) which is incorporated herein by reference in its entirety. A sufficient amount of RNase-free DNase available from Promega, Corp. under the tradename “RQ1 RNase-free DNase” was then added to the extracted RNA from each mouse such that each respective product contained 200 ng of DNased-RNA using the procedure set forth in “RNase-free DNase” protocol published by Promega, Corp. (May, 1995). The resulting 200 ng of DNased-RNA was reverse transcribed (“RT”) using the procedure set forth in “Superscript II Reverse Transcriptase” a protocol published by Gibco-BRL (now Life Technologies, Inc.) (April 1992), using random hexamers as random primers which are commercially available from Life Technologies, Inc.

The resulting RT products were then amplified via a polymerase chain reaction (“PCR”) using about a 0.5 unit (per 100 μl reaction) of a thermostable DNA polymerase which is commercially available from Perkin-Elmer-Cetus Corporation under the tradename “Taq polymerase,” and about 0.1 μmol/reaction of mouse glyceraldehyde-3-phosphate-dehydrogenase (G3PDH) primers available from Clontech Laboratories, Inc. (“Clontech”), or primers as set forth in Table 4 (using the conditions in Table 4 or in accordance with the procedures set forth in the protocol accompanying the primers from Clontech).

Table 5 illustrates some of the DNA primers used, the amount of MgCl₂ required for the PCR reaction, and the length of the PCR cycle. Involucrin primers were as described in Marthinuss, et al., “Apoptosis in Pam212, an Epidermal Keratinocyte Cell Line: A Possible Role for bcl-2 in Epidermal Differentiation”, 6 Cell Growth Diff. 239-250 (1995) which is incorporated herein by reference in its entirety.

TABLE 4
DNA Primers Utilized in RT-PCR Assay

| | | Cycle | | Seq. |
|---|---|---|---|---|
| DNA Primer | MgCl₂ | (min) @ | Number of | ID |
| (See attached Sequence Listing) | (mM) | ° C. | cycles | No. |
| Transglutaminase sense | 2.5 | 1 @ 94; | 35 | 1 |
| 5′ AACCCCAAGT TCCTGAAG | | 2 @ 55; | | |
| | | 3 @ 72 | | |
| Transglutaminase antisense | 2.5 | 1 @ 94; | 35 | 2 |
| 5′ TTTGTGCTGG GCCACTTC | | 2 @ 45; | | |
| | | 3 @ 72 | | |
| Elastin sense | 5 | 1 @ 94; | 35 | 3 |
| 5′ TAAGGCAGCC AAATATGGTG | | 2 @ 45; | | |
| | | 3 @ 72 | | |
| Elastin antisense | 5 | 1 @ 94; | 35 | 4 |
| 5′ ACCTGGATAA ATGGGAGAAA G | | 2 @ 55; | | |
| | | 3 @ 72 | | |

When necessary for better visualization, the resulting PCR products were precipitated with ethanol according to well-known procedures. When primers for G3PDH were used, only 10% of the PCR reaction products were used.

The PCR products were then analyzed on 2% agarose/ethidium bromide gels according to methods well-known in the art in order to compare the level of expression of certain genes in skins of trypsin-treated and untreated mice. An RNA sample from the skin of a Rhino mouse that was not reverse-transcribed was used as a negative control for each PCR amplification. An RNA sample from the skin of a six month old Rhino mouse was used as a positive control when positive controls were not commercially available. The results of the gel analysis showed that the migration of the RT-PCR products on the gels was always identical to that of the positive controls, and to that of the reported amplimer sizes.

The relative quality of each respective RT-PCR reaction product was then compared by analyzing the mRNA level of G3PDH, a “housekeeping” gene, in each respective product. As illustrated in FIG. 6, G3PDH gene expression was found to be similar in all samples examined, which thereby enabled the analysis of the relative levels of gene expression for the desired genes.

Transglutaminase, an enzyme involved in the cross linking and formation of apoptotic bodies, displayed high mRNA levels in control animals, and was reduced to below detection level with increasing concentrations of trypsin. This shows that trypsin restored utriculi homeostasis and eliminated abnormally high levels of apoptosis in the follicular epithelium.

Elastin mRNA increased following treatment with increasing concentrations of trypsin. Therefore, new elastin is expressed following trypsin treatment which results in increased skin elasticity, as described in Example 5.

The level of involucrin, a marker of epidermal differentiation, was increased following trypsin treatment in a dose dependent manner. This indicates that normal epidermal turnover and differentiation were restored. Thus, trypsin restores the balance of epidermal differentiation as shown in Example 8.

This Example showed that the effect of trypsin on Acne Vulgaris and its anti-aging abilities may be understood by examination of the expression pattern of a series of genes over a range of trypsin concentrations. Trypsin-induced changes in mRNA levels were clearly evidenced, indicating a regulatory role for trypsin in PCD, apoptosis, elastin expression, and epidermal differentiation.

### Example 10

### Use of Compositions Containing Trypsin and All-Trans Retinoic Acid

Glycerol dilaurate/cholesterol/polyoxyethylene-10-stearyl ether liposomes are prepared in accordance with the procedures set forth in Niemiec, wherein the constituent compounds of the liposomes are in a ratio of about 58:15:27, respectively. Prior to mixing the lipid and water phases to form the liposomes of Niemiec, 0.1% (w/v) ascorbic acid is added to the water phase, and the ingredients listed in Table 5 are added to the lipid phase of the composition. The final pH of this composition is adjusted to a range of 4 to 7, and prefereably from 4.5 to 5.5 with a suitable buffer.

TABLE 5
Ingredients Added to the Lipid Phase

| Ingredient | % (w/v) |
|---|---|
| Tretinoin | 0.01 |
| Methyl Paraben | 0.10 |
| Propyl Paraben | 0.02 |
| Butylated Hydroxytoluene | 0.05 |

A second composition, which comprises 1.0 g trypsin disolved in a 0.05M Hepes buffer, at pH 7.4 (q.s. to 100 ml), is added to the liposome composition in a ratio of about 1 part of the second composition for every 8 parts of the liposome composition. This final composition is suitable for immediate topical application.

We claim:

1. A method for treating Acne Vulgaris and/or for producing anti-aging effects on the surface of the skin comprising topically applying to the skin of a mammal an effective amount of a topically active composition comprising a first topically active agent.

2. The method of claim 1 wherein the first topically active agent is a protease.

3. The method of claim 2 wherein the first topically active agent is a serine protease.

4. The method of claim 3 wherein the first topically active agent is selected from trypsin, tryptase, carboxypeptidase-Y, protease IV, subtilysin or mixtures thereof.

5. The method of claim 4 wherein the first topically active agent is trypsin.

6. The method of claim 5 wherein the first topically active agent is present in an amount, based upon the total volume of the topically active composition, of from about 0% (w/v) to 5% (w/v).

7. The method of claim 6 wherein the first topically active agent is present in an amount, based upon the total volume of the topically active composition, of from about 0.01% (w/v) to about 1% (w/v).

8. The method of claim 1 wherein said topically active composition further comprises a pharmaceutically or cosmetically acceptable vehicle.

9. The method of claim 8 wherein said pharmaceutically or cosmetically acceptable vehicle is a liposome or mixture thereof.

10. The method of claim 9 wherein said liposome is non-ionic.

11. The method of claim 10 wherein said liposome is comprised of:
a) glycerol dilaurate, glycerol distearate, or a mixture thereof;
b) cholesterol, or a compound having a steroid backbone as found in cholesterol or a mixture thereof; and
c) a fatty acid ether having from about 12 to about 18 carbon atoms or a mixture thereof.

12. The method of claim 11 wherein said liposome is comprised of:
a) glycerol dilaurate;
b) cholesterol; and
c) polyoxyethylene-10-stearyl ether.

13. The method of claim 11 wherein the components of said liposome are present in a ratio of about 53:10:22 to about 63:20:32, respectively.

14. The method of claim 8 wherein said pharmaceutically or cosmetically acceptable vehicle is present in an amount, based upon the total volume of said topically active composition, of from about 0 mg/mL to about 100 mg/mL.

15. The method of claim 1 wherein the composition further comprises other ingredients such as moisturizers, cosmetic adjuvants, anti-oxidants, surfactants, foaming agents, conditioners, humectants, fragrances, viscosifiers, buffering agents, sunscreens, colorants, preservatives, and the like.

16. A method for treating Acne Vulgaris and/or for producing anti-aging effects on the surface of the skin comprising topically applying to the skin of a mammal an effective amount of:
a) a first topically active agent; and
b) an effective amount of a second topically active agent.

17. The method of claim 16 wherein the first topically active agent is a protease.

18. The method of claim 17 wherein the first topically active agent is a serine protease.

19. The method of claim 18 wherein the first topically active agent is selected from trypsin, tryptase, carboxypeptidase-Y, protease IV, subtilysin or mixtures thereof.

20. The method of claim 19 wherein the first topically active agent is trypsin.

21. The method of claim 20 wherein the first topically active agent is present in an amount of from about 0% (w/v) to 5% (w/v).

22. The method of claim 21 wherein the first topically active agent is present in an amount of from about 0.01% (w/v) to about 1% (w/v).

23. The method of claim 16 wherein said second topically active agent is a retinoid.

24. The method of claim 23 wherein said second topically active agent is selected from retinoic acids, vitamin A alcohol, vitamin A aldehyde, retinyl acetate, retinyl palmitate, or other derivatives, analogs or mixtures thereof.

25. The method of claim 24 wherein said second topically active agent is all-trans retinoic acid.

26. The method of claim 24 wherein the second topically active agent is present in an amount of from about 0.0001% (w/v) to about 0.5% (w/v).

27. The method of claim 26 wherein the second topically active agent is present in an amount of from about 0.001% (w/v) to about 0.025% (w/v).

28. The method of claim 16 further comprising a pharmaceutically or cosmetically acceptable vehicle.

29. The method of claim 28 wherein said pharmaceutically or cosmetically acceptable vehicle is a liposome or mixture thereof.

30. The method of claim 29 wherein said liposome is non-ionic.

31. The method of claim 30 wherein said liposome is comprised of:
a) glycerol dilaurate, glycerol distearate, or a mixture thereof;
b) cholesterol, or a compound having a steroid backbone as found in cholesterol or a mixture thereof; and
c) a fatty acid ether having from about 12 to about 18 carbon atoms or a mixture thereof.

32. The method of claim 31 wherein said liposome is comprised of:
a) glycerol dilaurate;
b) cholesterol; and
c) polyoxyethylene-10-stearyl ether.

33. The method of claim 31 wherein the components of said liposome are present in a ratio of about 53:10:22 to about 63:20:32, respectively.

34. The method of claim 28 wherein said pharmaceutically or cosmetically acceptable vehicle is present in an amount, based upon the total volume of said topically active composition, of from about 0 mg/mL to about 100 mg/mL.

35. The method of claim 16 further comprising other ingredients such as moisturizers, cosmetic adjuvants, anti-oxidants, surfactants, foaming agents, conditioners, humectants, fragrances, viscosifiers, buffering agents, sunscreens, colorants, preservatives, and the like.

36. The method of claim 16 wherein the first topically active agent is applied to the skin of a mammal simultaneously with the second topically active agent.

37. The method of claim 16 wherein the first topically active agent is applied to the skin of a mammal at a time other than simultaneously with the second topically active agent.

38. A pharmaceutical or cosmetic composition comprising:
a) a first topically active agent; and
b) a second topically active agent.

39. The pharmaceutical or cosmetic composition of claim 38 wherein the first topically active agent is a protease.

40. The pharmaceutical or cosmetic composition of claim 39 wherein the first topically active agent is a serine protease.

41. The pharmaceutical or cosmetic compositiaon of claim 40 wherein the first topically active agent is selected from trypsin, carboxypeptidase-Y, protease IV, subtilysin or mixtures thereof.

42. The pharmaceutical or cosmetic composition of claim 41 wherein the first topically active agent is trypsin.

43. The pharmaceutical or cosmetic composition of claim 42 wherein the first topically active agent is present in an amount, based upon the total volume of the topically active composition, of from about 0% (w/v) to 5% (w/v).

44. The pharmaceutical or cosmetic composition of claim 43 wherein the first topically active agent is present in an amount, based upon the total volume of the topically active composition, of from about 0.01% (w/v) to about 1% (w/v).

45. The pharmaceutical or cosmetic composition of claim 38 wherein said second topically active agent is a retinoid.

46. The pharmaceutical or cosmetic composition of claim 45 wherein said second topically active agent is selected from retinoic acids, vitamin A alcohol, vitamin A aldehyde, retinyl acetate, retinyl palmitate, or other derivatives, analogs or mixtures thereof.

47. The pharmaceutical or cosmetic composition of claim 46 wherein said second topically active agent is all-trans retinoic acid.

48. The pharmaceutical or cosmetic composition of claim 46 wherein the second topically active agent is present in an amount, based upon the total volume of the topically active composition, of from about 0.0001% (w/v) to about 0.5% (w/v).

49. The pharmaceutical or cosmetic composition of claim 48 wherein the second topically active agent is present in an amount, based upon the total volume of the topically active composition, of from about 0.001% (w/v) to about 0.025% (w/v).

50. The pharmaceutical or cosmetic composition of claim 48 wherein said topically active composition further comprises a pharmaceutically or cosmetically acceptable vehicle.

51. The pharmaceutical or cosmetic composition of claim 50 wherein said pharmaceutically or cosmetically acceptable vehicle is a liposome or mixture thereof.

52. The pharmaceutical or cosmetic composition of claim 51 wherein said liposome is non-ionic.

53. The pharmaceutical or cosmetic composition of claim 52 wherein said liposome is comprised of:
a) glycerol dilaurate, glycerol distearate, or a mixture thereof;
b) cholesterol, or a compound having a steroid backbone as found in cholesterol or a mixture thereof; and
c) a fatty acid ether having from about 12 to about 18 carbon atoms or a mixture thereof.

54. The pharmaceutical or cosmetic composition of claim 53 wherein said liposome is comprised of:
a) glycerol dilaurate;
b) cholesterol; and
c) polyoxyethylene-10-stearyl ether.

55. The pharmaceutical or cosmetic composition of claim 53 wherein the components of said liposome are present in a ratio of about 53:10:22 to about 63:20:32, respectively.

56. The pharmaceutical or cosmetic composition of claim 50 wherein said pharmaceutically or cosmetically acceptable vehicle is present in an amount, based upon the total volume of said topically active composition, of from about 0 mg/mL to about 100 mg/mL.

57. The pharmaceutical or cosmetic composition of claim 38 wherein the composition further comprises other ingredients such as moisturizers, cosmetic adjuvants, anti-oxidants, surfactants, foaming agents, conditioners, humectants, fragrances, viscosifiers, buffering agents, sunscreens, colorants, preservatives, and the like.
